(19) United States
(12) Patent Application Publication
(10) Pub. No.: US 2024/0216623 A1
(43) Pub. Date: Jul. 4, 2024

(54) VALVED HOLDING CHAMBER WITH EXHALATION FILTER

(71) Applicant: Flexicare (Group) Limited, Mountain Ash (GB)

(72) Inventors: Michael NEWHOUSE, Hamilton (CA); Akanksha HANDE, Somerset, NJ (US); Rohinton TODDYWALA, Somerset, NJ (US)

(21) Appl. No.: 17/995,614

(22) PCT Filed: Apr. 5, 2021
(86) PCT No.: PCT/US21/25751

§ 371 Date: Dec. 20, 2023

Related U.S. Application Data
(60) Provisional application No. 63/060,927, filed on Aug. 4, 2020.
(60) Provisional application No. 63/006,055, filed on Apr. 6, 2020.

(51) Int. Cl. A61M 15/00; A61M 16/06

(52) U.S. Cl. CPC A61M 15/0086; A61M 15/0013; A61M 15/0021; A61M 15/009; A61M 16/06

(57) ABSTRACT

A valved holding chamber is provided for the administration of drugs to a patient by inhalation from a metered dose inhaler having an exhalation filter on an exhalation pathway adapted to trapping aerosols and droplets in exhaled air. The exhalation filter may be angled at an approximately 45° angle away from the face of the patient. The exhalation filter prevents transmission of airborne diseases such as COVID-19 or influenza, or drug particles that could be sensitizing agents for caregivers or others in the patient's environment.

### CROSS REFERENCE TO RELATED APPLICATION

This patent application claims priority to United States Patent Applications U.S. 63/006,055 filed Apr. 6, 2020, and U.S. 63/060,927 filed Aug. 4, 2020, the contents of each of which are incorporated by reference.

### FIELD OF THE INVENTION

This invention relates to the administration of drugs by inhalation with a metered dose inhaler and a valved holding chamber, wherein an exhalation filter on the chamber prevents contamination of the surrounding environment with infectious disease particles or sensitizing agents by trapping exhaled droplets from escaping the chamber.

### BACKGROUND

This invention addresses the problem of administering drugs from a pressurized metered dose inhaler (MDI) to persons who may have a communicable disease, for example influenza or COVID-19 that is transmitted by viral particles in aerosols or droplets in exhaled air. The exhalation from such infected persons is dangerous, since these aerosols or droplets can remain in suspended in the environment surrounding an infected person and can be inhaled by nearby people causing infection and thereby transmitting the disease. This is a particular problem for people in need of an inhaled drug (for example, albuterol/salbutamol) administered with a valved holding chamber (VHC). These patients, if infected with a contagious disease, can easily spread a contagion while exhaling during the use of a valved holding chamber.

In a similar fashion, there may be certain drugs administered with an MDI that are sensitizing to the patient or other surrounding persons if leaked or exhaled as aerosols into air nearby during the use of an MDI.

Accordingly, a means for preventing potentially infectious aerosols or drug substances from escaping to the environment nearby people using and MDI and VHC would be a desirable advance in the technology of administering drugs with an MDI and VHC.

The types of drugs used with a VHC can eliminate or reduce the need for more aggressive interventions, such as intubation and ventilators. At the same time, it is not convenient to use conventional interventions (such as face masks) to prevent expulsion of infectious agents from infected persons while simultaneously using a valved holding chamber.

VHCs were designed to assist patients with airflow obstruction to use a pressurized metered dose aerosol generator (MDI) more effectively by dissociating, for a few seconds, the aerosol discharge and inhalation and thus ensure delivery of aerosols to the airways such as, for example, inhaled bronchodilators, corticosteroids, and other medications without the need to coordinate breathing with the discharge of the drug substance from the MDI.

Aerosolized drugs are important medicaments for the treatment of asthma, chronic obstructive pulmonary disease (COPD), other respiratory diseases, and even other non-respiratory conditions, where delivery of a drug substance to the lungs is desired. Drugs delivered directly to the lungs may act locally in the lungs or be absorbed in the lungs for delivery elsewhere in the body. By the term “aerosolized drugs” is meant a suspension of fine solid or liquid drug substance in air that is intended for delivery by inhalation to the lungs of a patient in need of such drug. The term “atomized” is synonymous here with “aerosolized.”

A frequently used and inexpensive source of aerosolized drugs are metered dose inhalers (MDI's). They are extremely popular because of their ease of use, and because they can efficiently deliver aerosolized medication directly to the lungs, which is highly advantageous in respiratory conditions. MDIs consist of a pressurized canister containing a liquid or powdered drug product and a propellant, and include an actuation device, typically a Meshberg valve, and a valve stem as outlet. There is also typically an adapter with a mouthpiece. The valve stem is seated in a receptacle in the adapter. The valve stem and valve dispense a dose of the drug when the canister is depressed within the adapter. In a simple embodiment, the patient uses the mouthpiece of the adapter directly to inhale medication. A feature of these devices is that the patient must coordinate an inhalation with actuating the MDI. This coordination is a problem for many patients. Additionally, mouth or throat irritation, hoarseness and fungal infection can be a problem due to deposition of a large proportion of the drug or propellant particles in the mouth or throat, rather than the lungs.

VHC's, also known as “valved aerosol reservoirs” or “spacers,” can be coupled to MDI's and are well known in the art as having certain advantages. VHC's provide two principal benefits to putting the outlet of an MDI directly in the mouth. In a first advantage, a VHC may trap larger particles in the drug plume ejected from the MDI. These large particles lodge in the mouth or throat of the user if the MDI is inserted directly in the mouth. The use of VHC's results in less deposition of drug in the mouth and throat, which is undesirable, and improved delivery of the aerosolized drug to the lungs. In a second advantage, it is not necessary to coordinate breathing and actuation of the MDI with a VHC in use. The drug aerosol can remain suspended in the chamber for several seconds, and this delay may even have the advantage of allowing larger particles to sediment gravitationally or by means of static attraction out of the plume.

In some patient populations, the use of a VHC is mandatory. A simple spacer device is disclosed, for example, in WO 2004/091704.

In another aspect, a chamber may have a one-way inhalation valve, as disclosed for example in U.S. Pat. Nos. 5,012,804; 5,042,467; and 6,026,807. By the use of an inhalation valve, the user does not need to coordinate their inhalation with the source of aerosolized drug, such as an actuation of an MDI. This is important, for example, for inexperienced users, incompetent users, or children. The aerosol plume from the MDI can remain suspended within the chamber for up to 10 seconds before inhalation and deliver an effective dose to the lungs even if the patient exhales prior to inhaling.

In another aspect, a chamber with a one-way inhalation valve can be used with an inhalation mask. With an inhalation mask, the patient does not need to put their lips around a mouthpiece. This is particularly useful with small children or incompetent patients. Such masks have been disclosed, for example, in U.S. Pat. No. 5,645,049.

Attempts to minimize the spread of COVID-19 have involved isolating or quarantining much of the population in most countries. It is now appreciated that much of the person-to-person spread of the disease results from droplet nuclei and aerosols produced from exhalations of infected people, including routine breathing, speaking, coughing, and sneezing.¹ Accordingly, the use of face coverings that cover the nose and mouth have been recommended to trap exhaled particles and minimize the likelihood of others inhaling droplets.

Many of the persons afflicted with COVID-19 and other highly communicable diseases have airflow obstruction due to the viral bronchitis/bronchiolitis caused by the disease, or the patients may have previous asthma, COPD, bronchopulmonary dysplasia, or cystic fibrosis (CF) co-morbidity. Such patients are frequently administered bronchodilators, corticosteroids, muscarinic antagonists, and other drugs or combinations thereof to relieve airflow obstruction. Inhaled aerosols are frequently administered using MDIs with a VHC, which allows for consistent dosing and more efficient drug delivery in most scenarios. Moreover, patients may be advised, if they are able to follow instructions, to inhale as deeply as they can when using an MDI, with or without a VHC, to deliver drug as deep as possible into the lungs. Thus, the exhaled air from patients using a VHC, which may include coughing, can expel infectious droplets into the surrounding environment if the patient has a communicable disease spread by viral particles in exhaled air such as COVID-19 or influenza.

Thus, the problem addressed in this invention is the danger posed by the exhalation to open air from patients using a VHC, which is expected to expel droplets of potentially infectious exhalate. Earlier studies of metered dose inhalers using Tc99^{M} radio-labelled bronchodilator aerosols by means of scintigraphy showed mass balance determinations in the development of a VHC (“AeroChamber®”). It was found that about 90% of the drug plume from the MDI is delivered to the VHC prior to inhalation. About 20-30% of the drug remains suspended in the VHC and is drawn into the patient's mouth during an inhalation. This is the respirable portion of the aerosol plume. The mass median aerodynamic diameter of the respirable portion of the aerosol plume is about 5 μm or less. Ideally, the mass median aerodynamic diameter of inhaled aerosol is around 2 μm or less for optimal delivery to the lungs.² Upon inhalation, about one-third of the inhaled aerosol is deposited in the upper respiratory tract, leaving about 15-20% of the original dose that is delivered to the airways below the larynx and within the lungs. On average, about 3% of the lower respiratory tract dose is exhaled.

While this degree of exhalate is normally of no consequence, in patients with a highly infectious airborne disease (such as COVID-19) infectious droplets can be expelled into the environment in the vicinity of the patient, and pose a danger of exposing caregivers, other nearby patients in an emergency department, and bystanders, to dangerous contagions.

### SUMMARY OF THE INVENTION

In order to address the concern about exhaled air containing potentially infectious bacterial or viral particles from patients using a valved holding chamber, a valved holding chamber is provided for the administration of drugs by inhalation through a mouthpiece or a mask from a metered dose inhaler, wherein the chamber has an exhalation filter on an exhalation pathway adapted to trapping viral, bacterial, or chemical particles in exhaled air. In an embodiment, the exhalation filter is angled at an approximately 45° angle away from the face of the patient. In an embodiment, the exhalation filter prevents transmission of airborne diseases such as COVID-19.

Thus, in an embodiment, a valved holding chamber is provided for the administration of drugs from a metered dose inhaler (MDI) to a patient through a mouthpiece. The chamber may have a generally cylindrical configuration having a distal end adapted to engaging an MDI, and a proximal end having a mouthpiece, wherein the MDI, when activated, ejects an aerosolized drug plume into the chamber and wherein the respirable portion of the aerosolized drug remains suspended in the chamber prior to an inhalation by the patient. The proximal end of the chamber comprises an airway including a mouthpiece for insertion into the mouth of the patient, and wherein the airway includes a one-way inhalation valve that permits air and aerosolized drug to enter the airway and mouthpiece from the chamber during an inhalation, and wherein the inhalation valve is otherwise closed and prevents air and aerosolized drug from entering the airway. The one-way valve also prevents exhaled air from entering the chamber. The airway may include an exhalation pathway having a one-way exhalation valve that only permits the passage of exhaled air during an exhalation. The exhalation pathway may have an exhalation channel external to the airway pathway and the generally cylindrical chamber, such that the exhalation channel includes an exhalation filter positioned at an approximately 45° angle from a longitudinal axis of the chamber and airway pathway, wherein the angle is away from the face of the patient, and wherein all exhaled air passes through the exhalation filter, and wherein no external air passes through the exhalation filter when the patient is not exhaling.

In an alternative embodiment, a valved holding chamber for the administration of a drug product from a metered dose inhaler (MDI) to a patient using an inhalation mask is provided, having a generally cylindrical chamber having a distal end adapted to engaging an MDI, and a proximal end having an inhalation mask, wherein the MDI, when activated, ejects an aerosolized drug plume into the chamber and wherein a respirable portion of the aerosolized drug remains suspended in the chamber prior to inhalation by the patient. The proximal end of the chamber may have an airway including an inhalation mask fitting over the nose and mouth of the patient, and wherein the airway includes a one-way inhalation valve that permits air and aerosolized drug to enter the airway and mask from the chamber during an inhalation and wherein the inhalation valve is otherwise closed and prevents air and aerosolized drug from entering the airway. The airway may include a tubular exhalation pathway branching off the airway and interposed approximately midway between the chamber and mask, wherein the exhalation pathway is at an approximately 45° angle towards the chamber. The exhalation pathway at the approximately 45° angle includes a one-way exhalation valve and an exhalation filter, and wherein all exhaled air passes through the exhalation filter, and wherein no external air passes through the exhalation filter when the patient is not exhaling.

### DESCRIPTION OF THE DRAWINGS

FIG. 1A is a perspective partially transparent view of an embodiment of the inventive apparatus for use with a mouthpiece.

FIG. 1B is a view of the exhalation valve of the embodiment from FIG. 1A.

FIG. 1C is a cross-section of the exhalation filter section of the embodiment from FIG. 1A.

FIG. 2 is a cross-section view of the embodiment from FIG. 1A showing airflow during the inhalation phase of a breath.

FIG. 3 is a cross-section view of the embodiment from FIG. 1A showing airflow during the exhalation phase of a breath.

FIG. 4 is a perspective partially transparent view showing a valved holding chamber with a “SootherMask”® inhalation mask.

FIG. 5 is a perspective partially transparent view showing a valved holding chamber with a “InspiraMask”® inhalation mask.

FIG. 6 is a cross section view of the embodiment of FIG. 4 illustrating the dead space in the airway.

FIG. 7 s a cross section view of the embodiment of FIG. 5 illustrating the dead space in the airway.

### DETAILED DESCRIPTION

In an embodiment, a valved holding chamber (VHC) is provided having a microbial and viral exhalation filter that traps potentially infectious aerosol being exhaled by a person, also termed a “patient,” using the VHC to receive an aerosolized drug by inhalation. Others have previously disclosed exhalation filters (but with substantial difference from the inventive structures) in WO 02/04054 and US2005/0217667.

In the inventive apparatus, a modified VHC is provided. The VHC may have a generally cylindrical chamber and a distal end adapted to support and hold a metered dose inhaler (MDI). The proximal end of the VHC may have an airway and a one-way inhalation valve, such that when the patient inhales, air and aerosolized drug in the interior of the VHC is drawn into the patient's lungs, but air from the patient on exhalation is blocked from entering the interior of the VHC.

In an embodiment, the inventive apparatus includes an exhalation filter on the VHC apparatus to prevent infectious particles or sensitizing agents in the exhaled air from the patient from escaping into the environment around the patient, and potentially exposing other people near the patient, for example caregivers, family members, and nearby patients in an emergency department, to infectious or sensitizing particles suspended in the air.

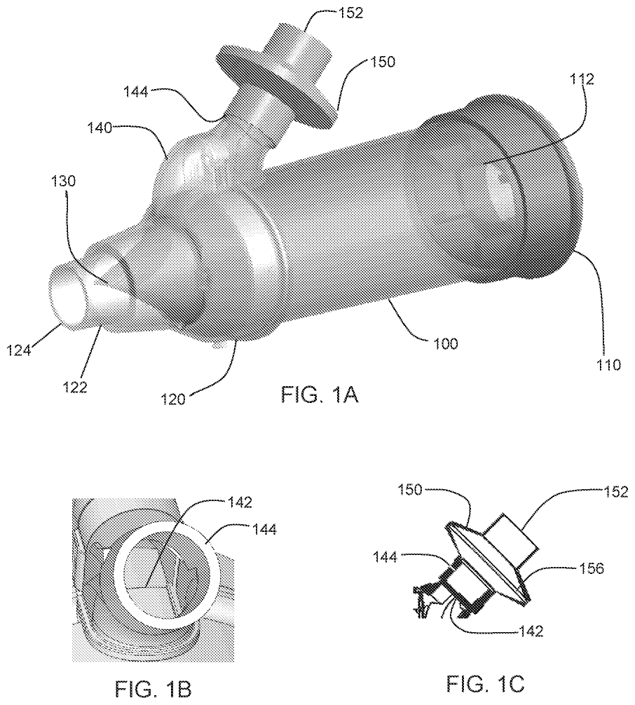

An embodiment of the inventive apparatus is shown in FIGS. 1-3. FIG. 1A shows a perspective x-ray view of an embodiment of the inventive apparatus using a conventional mouthpiece that interfaces with the patient. VHC body 100 has a distal end 110 having a collar 112 that supports an MDI. The proximal end 120 of the VHC is a tapered cap defining an airway 122 that may include within a one-way inhalation valve 130. In the embodiment shown, valve 130 may be a duck-bill style valve. Other valve styles are possible here, for example, a single flap valve. The end of the airway is mouthpiece 124, that a patient can insert into their mouth and wrap their lips around during use of the VHC.

The inventive apparatus may include an exhalation pathway 140 branching off airway 122. Exhalation pathway 140 may lead to a one-way exhalation valve 142. Thus, as the patient exhales, the exhaled gases are blocked from reentering the chamber 100 by the one-way inhalation valve and are directed to exhalation valve 142 as the only exit path. Likewise, during an inhalation portion of the breathing cycle, when the mouthpiece is 124 is secured in the mouth and lips of a patient, all air to the patient passes through the interior 100 of the VHC, so any aerosolized drug in the interior of VHC 100 is inhaled, and no external air can pass through the exhalation valve 142.

In an embodiment as shown in FIG. 1B, exhalation valve 142 may be a pair of flexible rubber flaps configured so they can only flex outward to permit exhaled air to escape and preventing the passage of air during an inhalation. Other embodiments of an exhalation valve are possible, for example a single flap or a duck-bill valve arrangement.

The exhaled air, after passing through valve 142, continues through path 144 which may be a collar that supports exhalation filter 150. In an embodiment, the filter may be, for example, the Ventlab/Sun Med #FH603003. Although this is characterized as a “bacterial filter,” this filter can trap exhaled droplets that are believed to convey infectious airborne agents for diseases such as COVID-19 or influenza. The bacterial/viral filter valve may be removable and replaceable. In an embodiment, the bacterial/viral filter is not washable while the chamber can be washed and sterilized. In an embodiment, the bacterial/viral filter may be replaced on a set schedule after a predetermined number of treatments to prevent contamination. In an embodiment, the bacterial/viral filter may be replaced for each individual patient.

In an embodiment, the filter assemblies are angled so that the air outlet 152 is directed away from the face of the patient. This is shown in the drawings. At the same time, the entire apparatus has a compact, simple profile. Angling the filter away from the face makes a more comfortable experience for the patient, minimizing a plastic object that might be right in front of the eyes or touching the face.

FIG. 1B is a perspective view of the one-way exhalation valve 142 in the exhalation path. Also shown is collar 144 that may support exhalation filter 150. FIG. 1C is a cross section view of the filter and exhalation path. Filter apparatus 150 includes a filter membrane 156. Membrane 156 is the active region that traps aerosols in the exhaled air from exiting the apparatus through outlet 152.

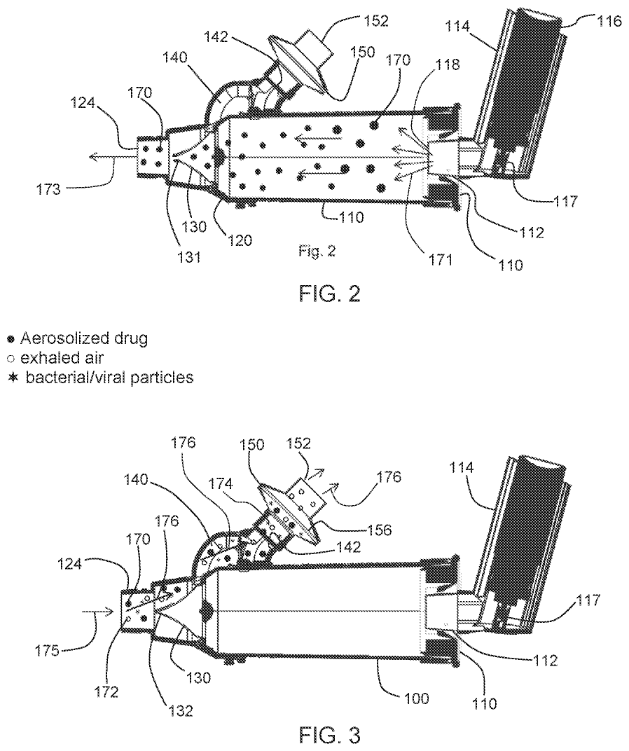

FIGS. 2 and 3 demonstrate the inventive apparatus in operation during inhalation and exhalation portions of a breathing cycle. FIG. 2 is a cross-section view showing the interior 100 of the VHC with suspended aerosolized drug 170 during an inhalation. One of the advantages of a VHC when used with an MDI drug is that the patient need not coordinate breathing and the activation of the MDI. The activation of the MDI can be as much as 10 seconds prior to an inhalation. FIGS. 2 and 3 show the MDI having canister 116 that holds a drug under pressure, MDI body 114, MDI valve 117, and MDI outlet 118. The output of aerosolized drug 170 is indicated by arrows 171. When the patient inhales (FIG. 2), the respirable portion of the aerosolized drug and air in the chamber 100 is drawn through one-way valve 130, shown in the open position (131). Air and aerosolized drug are then drawn into the mouth of the patient, shown by arrow 173. By the term “respirable portion,” it is meant aerosol particles with a mass median aerodynamic diameter of 5 μm or less. Even smaller particles, such as 2 μm or less are preferable as being more likely to be deposited into the small airways of the lungs.

During the exhalation portion of a breathing cycle (FIG. 3), exhaled gases enter mouthpiece 124 as shown by arrow 175. The exhaled gases include some amount of aerosolized drug that was not absorbed by the lungs, exhaled air, and water vapor which may include bacterial and viral particles 174 if the patient is infected with a disease transmissible through the air. The exhaled gases are forced into exhalation pathway 140, and pass through exhalation valve 142. The path of the exhalation gas flow is shown by arrows 176. After passing through the exhalation valve 142, the exhalation gases flow through bacterial/viral filter 150 and exit to the atmosphere through port 152.

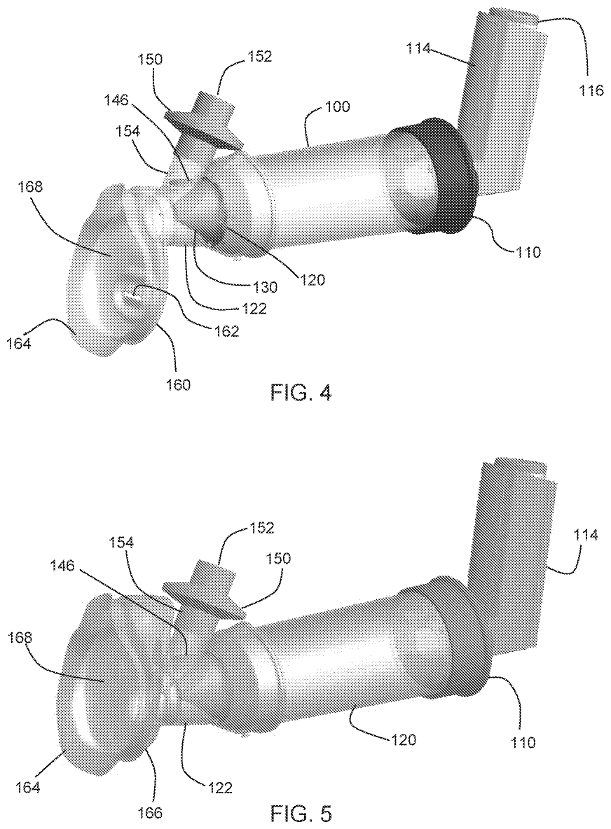

FIGS. 4 and 5 show an embodiment of the inventive apparatus with an inhalation mask instead of mouthpiece 124. Two variations of inhalation masks are depicted. FIG. 4 shows the use of a “SootherMask”® (160), a mask specifically for infants and small children with a slot 162 in the mask for the insertion of a pacifier (not shown), i.e., a plastic sucking toy, that the infant can put in their mouth while using the mask. This type of toy may make the infant feel more comfortable while using the mask, and the sucking action may tend to draw the mask towards the face gently, which is desirable. The airway port into the SootherMask is across from the nose of the child, since infants and small children are preferential nasal breathers.

FIG. 5 shows the use of an “InspiraMask”® (166) in the inventive apparatus, which is also designed for use by infants or small children. Both the Soothermask and InspiraMask have a contoured shape with a lip designed to fit the contours of children‘s’ faces.³ In an embodiment, the contoured shape and lip on the mask provides a tight fit and good seal around the face of the face of a child. Similar masks may provide a tight fit and good seal around the face of an older person. In addition, the shape of the interior surface 168 of both masks has been designed to minimize dead space in the mask.

Both the SootherMask and InspiraMask come in several different sizes. The SootherMask comes in small and medium sizes, and the InspiraMask comes in small, medium, and large sizes. Other mask embodiments are possible also.

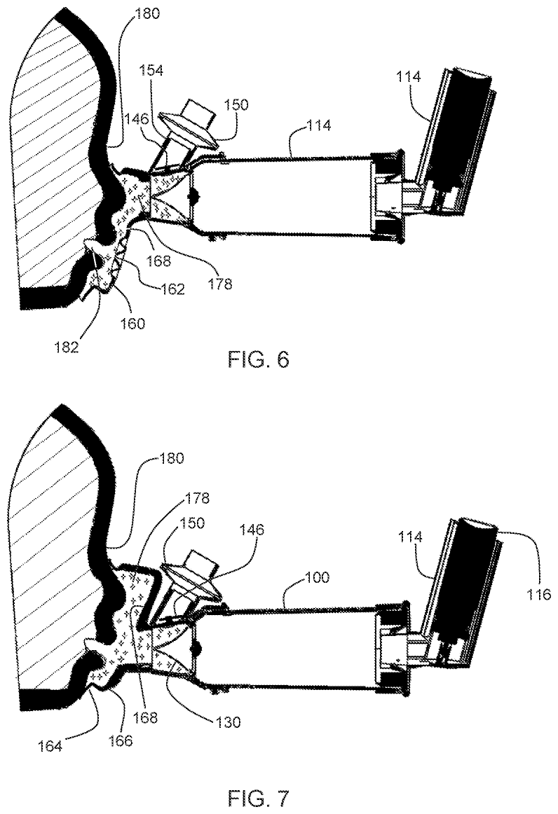

The embodiments in FIGS. 4 and 5 are shown with an exhalation path stem 154 branching off the airway 122. Stem 154 includes a one-way exhalation valve at the base of the stem (146 in FIGS. 6 and 7). Stem 154 defines an exhalation gas path after the exhalation valve. Stem 154 also acts as a collar to support exhalation filter 150.

Thus, in FIGS. 4 and 5, as the patient exhales, inhalation valve remains closed, exhalation valve 146 opens, and the exhalation gases are vented to exhalation filter 150 and then to the atmosphere through port 152.

In an embodiment, a feature of the mask embodiments as depicted in FIGS. 4-7 is that the dead space 178 between the inhalation valve 130, exhalation valve 146, and the patients face 180, is minimized (FIGS. 6-7). Minimal dead space in the mask has been shown to be an important parameter for children who have a low tidal volume in their breathing and in others with decreased compliance causing shallow breathing due to disease. In these patients, excessive dead space results in reduced delivery of aerosolized drug to the lungs.⁴ Aerosolized drug may remain suspended in any dead space, and the drug that remains in the dead space when an inhalation is complete will be expelled from the apparatus on exhalation, causing loss of drug. Thus, minimizing dead space in the apparatus is desirable. The dead-space volume (VD) of a face mask has thus been shown to critically affect aerosol delivery to infants and young children.

The inventors have measured the volume of the dead space 178 in the embodiments of FIGS. 4 and 5 using computer aided design (CAD) software. The following results shown in Table 1 were obtained.

TABLE 1
Dead Space in inhalation masks

| | Approximate Dead |
|---|---|
| Mask Style | Space Volume (mL) |
| InspiraMask small | 21 |
| InspiraMask medium | 41 |
| SootherMask small | 24 |
| SootherMask medium | 39 |

As shown by the Amirav and Newhouse paper (n. 4), these are significantly smaller volumes than in competitive masks.

Drawings Legend

| | |
|---|---|
| 100 | Generally cylindrical chamber |
| 110 | Distal end of chamber |
| 112 | Mount (collar) for MDI |
| 114 | MDI body |
| 116 | MDI cannister |
| 117 | MDI valve |
| 118 | MDI outlet |
| 120 | Proximal end of chamber |
| 122 | Airway |
| 124 | Mouthpiece |
| 130 | Inhalation valve - duckbill, closed |
| 131 | Inhalation valve open |
| 132 | Inhalation valve closed |
| 140 | Exhalation pathway on chamber |
| 142 | Exhalation valve on exhalation pathway |
| 144 | Exhalation path collar after exhalation valve |
| 146 | Exhalation valve on mask embodiment |
| 150 | Exhalation filter |
| 152 | Exhalation filter output - open to ambient atmosphere |
| 154 | Exhalation path (stem) on mask embodiment |
| 156 | Exhalation filter membrane |
| 160 | SootherMask ® |
| 162 | Slot in SootherMask |
| 166 | InspiraMask ® |
| 168 | InspiraMask/SootherMask interior surface of mask |
| 170 | Aerosolized drug |
| 171 | Aerosolized drug leaving MDI after activation |
| 172 | Exhaled air |
| 173 | Inhaled air direction |
| 174 | Bacteria/virus particles |
| 175 | Exhaled gas flow into mouthpiece |
| 176 | Exhaled gas flow in airway |
| 178 | Dead space in mask and airway |
| 180 | Child's face profile |
| 182 | Child's mouth in mask |

### REFERENCES AND NOTES

¹ M. Jayaweera et al., “Transmission of COVID-19 virus by droplets and aerosols: A critical review on the unresolved dichotomy,” Environ Res. 2020 September; 188: 109819. doi: 10.1016/j.envres.2020.109819; L. Morawska and J. Cao, “Airborne transmission of SARS-CoV-2: The world should face the reality,” Environ Int. 2020 June; 139: 105730. doi: 10.1016/j.envint.2020.105730
² S. Jabbal et al., “Does size really matter ?: Relationship of particle size to lung deposition and exhaled fraction,” J Allergy Clin Immunol, 2017, 139(6), 2013-2014, http://dx.doi.org/10.1016/j.jaci.2016.11.036
³ These masks are disclosed in US 2012/0318265 A1.
⁴ I. Amirav and M. Newhouse et al., “Computerized Dead-Space Volume Measurement of Face Masks Applied to Simulated Faces,” Resp. Care, 2015, 60(9), 1247-1251, DOI: 10.4187/respcare.03813.

1. A valved holding chamber for the administration of drugs from a metered dose inhaler (MDI) to a patient through a mouthpiece, comprising
a. a generally cylindrical chamber having a distal end adapted to engaging an MDI, and a proximal end having a mouthpiece, wherein the MDI, when activated, ejects an aerosolized drug plume into the chamber and wherein the respirable portion of the aerosolized drug remains suspended in the chamber prior to an inhalation by the patient;
b. wherein the proximal end of the chamber comprises an airway including a mouthpiece for insertion into the mouth of the patient, and wherein the airway includes a one-way inhalation valve that permits air and aerosolized drug to enter the airway and mouthpiece from the chamber during an inhalation, and wherein the inhalation valve is otherwise closed and prevents air and aerosolized drug from entering the airway;
c. wherein the airway includes an exhalation pathway having a one-way exhalation valve that only permits the passage of exhaled air during an exhalation;
d. wherein the exhalation pathway comprises a channel external to the airway pathway and the generally cylindrical chamber; and
e. wherein the exhalation channel includes an exhalation filter positioned at an approximately 45° angle from a longitudinal axis of the chamber and airway pathway, wherein the angle is away from the face of the patient, and wherein all exhaled air passes through the exhalation filter, and wherein no external air passes through the exhalation filter when the patient is not exhaling.

2. A valved holding chamber for the administration of a drug product from a metered dose inhaler (MDI) to a patient using an inhalation mask, comprising
a. a generally cylindrical chamber having a distal end adapted to engaging an MDI, and a proximal end having an inhalation mask, wherein the MDI, when activated, ejects an aerosolized drug plume into the chamber and wherein the respirable portion aerosolized drug remains suspended in the chamber prior to inhalation by the patient;
b. wherein the proximal end of the chamber comprises an airway including an inhalation mask fitting tightly over the nose and mouth of the patient, and wherein the airway includes a one-way inhalation valve that permits air and aerosolized drug to enter the airway and mask from the chamber during an inhalation and wherein the inhalation valve is otherwise closed and prevents air and aerosolized drug from entering the airway;
c. wherein the airway includes a tubular exhalation pathway branching off the airway and interposed approximately midway between the chamber and mask, wherein the exhalation pathway is at an approximately 45° angle towards the chamber; and
d. wherein the exhalation pathway at the approximately 45° angle includes a one-way exhalation valve and an exhalation filter, and wherein all exhaled air passes through the exhalation filter, and wherein no external air passes through the exhalation filter when the patient is not exhaling.

3. The valved holding chamber and inhalation mask of claim 2, wherein the mask as adapted for use with a child, wherein the dead space in the mask and airway has a volume according to the table:

| | Approx. Dead |
|---|---|
| Mask Style | Space Volume (mL) |
| InspiraMask small | 21 |
| InspiraMask medium | 41 |
| SootherMask small | 24 |
| SootherMask medium | 39 |

4. The valved holding chamber of claim 1 or 2, wherein the exhalation filter prevents the transmission of exhaled vapor and liquid from transmitting bacterial or viral infectious disease to the surrounding environment.

5. A method of administering drugs by inhalation from a metered dose inhaler (MDI) to a patient and preventing the transmission of exhaled droplets from the patient, comprising the valved holding chamber of claim 1 or 2, wherein an MDI is inserted in the distal end and activated, and the patient inhales the inhalable drug and exhales through the exhalation filter.

6. The administration of drugs by inhalation from a metered dose inhaler (MDI) to a patient and preventing the transmission of exhaled droplets from the patient, comprising the valved holding chamber of claim 1 or 2, wherein an MDI is inserted in the distal end and activated, and the patient inhales the respirable component of the inhalable drug and exhales through the exhalation filter, wherein the respirable component comprises aerosol with a mass median aerodynamic diameter of 5 μm or less.

7. An improved valved holding chamber for the administration of drugs from a metered dose inhaler (MDI) to a patient through a mouthpiece having
a. a generally cylindrical chamber having a distal end adapted to engaging an MDI, and a proximal end having a mouthpiece, wherein the MDI, when activated, ejects an aerosolized drug plume into the chamber and wherein a respirable portion of the aerosolized drug remains suspended in the chamber prior to an inhalation by the patient;
b. wherein the proximal end of the chamber comprises an airway including a mouthpiece for insertion into the mouth of the patient, and wherein the airway includes a one-way inhalation valve that permits air and aerosolized drug to enter the airway and mouthpiece from the chamber during an inhalation, and wherein the inhalation valve is otherwise closed and prevents air and aerosolized drug from entering the airway;
c. wherein the improvement comprises an exhalation pathway having a one-way exhalation valve that only permits the passage of exhaled air during an exhalation;
d. wherein the exhalation pathway comprises a channel external to the airway pathway and the generally cylindrical chamber; and
e. wherein an exhalation filter on the exhalation channel, wherein the exhalation filter is adapted to trapping bacterial or viral particles in exhaled air, wherein the exhalation filter is positioned at an approximately 45° angle from a longitudinal axis of the chamber and airway pathway, wherein the angle is away from the face of the patient, and wherein all exhaled air passes through the exhalation filter, and wherein no external air passes through the exhalation filter when the patient is not exhaling.

8. An improved valved holding chamber for the administration of a drug product from a metered dose inhaler (MDI) to a patient using an inhalation mask, having
a. a generally cylindrical chamber having a distal end adapted to engaging an MDI, and a proximal end having an inhalation mask, wherein the MDI, when activated, ejects an aerosolized drug plume into the chamber and wherein a respirable portion of the aerosolized drug remains suspended in the chamber prior to inhalation by the patient;
b. wherein the proximal end of the chamber comprises an airway including an inhalation mask fitting tightly over the nose and mouth of the patient, and wherein the airway includes a one-way inhalation valve that permits air and aerosolized drug to enter the airway and mask from the chamber during an inhalation and wherein the inhalation valve is otherwise closed and prevents air and aerosolized drug from entering the airway;
c. wherein the improvement comprises an exhalation channel comprising a tubular channel branching off the airway and interposed approximately midway between the chamber and mask, wherein the exhalation pathway is at an approximately 45° angle towards the chamber; and
d. wherein the exhalation pathway at the approximately 45° angle includes a one-way exhalation valve and an exhalation filter, wherein the exhalation filter is adapted to trapping bacterial or viral particles in exhaled air, and wherein all exhaled air passes through the exhalation filter, and wherein no external air passes through the exhalation filter when the patient is not exhaling.
